(19) United States
(12) Patent Application Publication
(10) Pub. No.: US 2025/0194941 A1
(43) Pub. Date: Jun. 19, 2025

(54) WIRELESS, BATTERYLESS BLOOD PRESSURE SENSOR IMPLANT

(71) Applicants: UNITED STATES GOVERNMENT as represented by THE DEPARTMENT OF VETERANS AFFAIRS, Washington, DC (US); CASE WESTERN RESERVE UNIVERSITY, Cleveland, OH (US)

(72) Inventors: Yaneev Hacohen, Cleveland, OH (US); Steve Majerus, Cleveland, OH (US)

(21) Appl. No.: 18/849,757

(22) PCT Filed: Mar. 24, 2023
(86) PCT No.: PCT/US2023/016217

§ 371 Date: Sep. 23, 2024

Related U.S. Application Data
(60) Provisional application No. 63/323,354, filed on Mar. 24, 2022.

(51) Int. Cl. A61B 5/0215; A61B 5/00; A61B 5/026; H01Q 1/36

(52) U.S. Cl. CPC A61B 5/0215; A61B 5/0031; A61B 5/026; A61B 5/6847; H01Q 1/362; A61B 2560/0219

(57) ABSTRACT

An implantable blood-flow sensor assembly for monitoring a portion disposed inside a user, including an antenna assembly, a flexible sensor assembly that is configured to be wrapped around the portion of the user, and microcontroller assembly that is operably connected to the antenna assembly and the flexible sensor.

### CROSS-REFERENCE TO RELATED APPLICATION

This application claims priority to and the benefit of the filing date of U.S. Provisional Patent Application No. 63/323,354, filed Mar. 24, 2022, the contents of which are incorporated herein by reference in their entirety.

### FIELD

The application is generally related to devices that are implantable into an individual, and, in particular, to implantable devices (e.g., implantable pressure and/or flow sensing devices) that are wireless and do not use batteries. Systems and methods for using the implantable devices are also disclosed.

### BACKGROUND

At least one goal of monitoring the flow of blood within vessels is to reduce the incidence of clotting, or thrombosis, which could cause major detrimental effects to tissue supplied by an occluded vessel. Monitoring is an essential clinical tool for numerous vascular conditions to identify or predict those who may benefit from prophylactic treatment or surgical planning. For example, patients with synthetic graft implantations, vessel transplants, and systemic blood-flow-related diseases would benefit from continuous monitoring of their blood flow and related pressure, as discussed below. Specifically, monitoring produces data that is easily gathered at a higher frequency (and lower cost) but is generally less precise than more costly approaches. In contrast, surveillance is targeted, and usually is more expensive such as the use of ultrasound to detect carotid arterial flow in patients at risk for stroke. One primary issue with surveillance is that it is often too costly for regular use, and that certain vascular conditions, such as stenosis formation, can occur quite rapidly. As a result, vascular surveillance programs are often unable to detect those at risk before a vascular condition becomes symptomatic. Pre-existing modalities of surveillance for blood flow can involve ultrasound, imaging, CT scans, or angiograms. These methods require doctors or other professional personnel to administer treatments, or require specialized equipment. Additionally, these methods are too costly for widespread use and regulated surveillance of all at-risk patients. Besides cost, some imaging modalities expose the patient to increased radiation, limiting widespread use. A dose equivalent to that of a single interventional fluoroscopy such as an angiogram has 250 to 3000 times the dose of a standard X-Ray. As a result, it is difficult to perform these surveillance scans frequently enough to detect underlying trends caused by ongoing issues. Therefore, various conditions would benefit from real-time monitoring of disease progression.

### SUMMARY

Embodiments of the disclosed blood pressure sensor implants demonstrate batteryless, implantable blood-flow sensor assemblies which use wireless power transfer (WPT) for communication and powering implanted electronics with an external transceiver. Preferably the WPT system uses a split-double helix antenna (DHA), enabling the formation of a cuff that can be slipped around a tubular structure in the body. The mutual inductance of the DHA is analytically modeled and validated for a range of DHA diameters. A radio-frequency identification (RFID) system enables WPT and data readout transfer to an external transceiver. In one embodiment, a sample rate of 12 Hz and reading distance of 3.5 cm can be achieved. The implantable DHA system is developed to wrap around vessels having diameters of 3 to 8 mm, although other diameters are possible, and couple to a strain-sensitive flexible pulsation sensor (FPS) formed of a carbon black-silicone nanocomposite. The FPS strain changes during pulsatile flow can be measured and wirelessly transmitted, enabling flow rate monitoring on a vascular phantom.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIGS. 1A and 1B are schematic representations of a primary coil and secondary coil during flux exposure;

FIG. 2 is a schematic representation of a transponder and transceiver placed for maximum flux transmission;

FIG. 3 is a schematic representation of an embodiment of an implantable blood-flow sensor assembly in accordance with the present invention;

FIGS. 4A and 4B are top plan views of an embodiment of an implantable blood-flow sensor assembly as shown in FIG. 3;

FIG. 5 is a schematic side view of a cell of the double helix antenna (DHA) of the sensor assembly shown in FIGS. 4A and 4B;

FIG. 6 is a schematic side view of the DHA of the sensor assembly shown in FIGS. 4A and 4B;

FIG. 7 is a schematic view of Celli and Cellj of the DHA of the sensor assembly shown in FIGS. 4A and 4B;

FIG. 8 is a schematic view of misaligned coils;

FIG. 9 is a schematic view of the offset of misalignment from Celli to Cellj, as shown in FIG. 7;

FIG. 10 is a schematic representation of the DHA and transmitter system of the sensor assembly, shown in FIGS. 4A and 4B;

FIG. 11 is a schematic representation of the DHA trace of the sensor assembly shown in FIGS. 4A and 4B;

FIG. 12 is a top plan view of the DHA trace of the sensor assembly shown in FIGS. 4A and 4B;

FIG. 13 is a perspective view of the DHA of the sensor assembly shown in FIGS. 4A and 4B, rolled into a cylindrical cuff;

FIG. 14 is a graphical representation of theoretical coupling coefficients for the DHA shown in FIG. 13;

FIG. 15 is a graphical representation of 15 and 20 mil spacing for DHAs of diameter 3, 4, and 5 mm;

FIG. 16 is a graphical representation of varying resistances of the DHAs as used in the sensor assembly shown in FIGS. 4A and 4B;

FIG. 17 is a top plan view of the DHA assembly and associated tuning capacitors of the sensor assembly shown in FIGS. 4A and 4B;

FIG. 18 is a graphical representation of calculated theoretical self-inductance versus measured values for DHAs of various diameters;

FIG. 19 is a circuit diagram showing the calculation of mutual inductance;

FIGS. 20A and 20B are graphical representations of measured versus calculated mutual inductance for various DHAs;

FIGS. 21A and 21B are graphical representations of measured versus calculated mutual inductance for various DHAs;

FIG. 22 is a block diagram of communication between an implantable blood-flow sensor assembly and corresponding transceiver of the present invention;

FIG. 23 is a schematic view of a radiofrequency integrated circuit (RFIC) of the sensor assembly shown in FIGS. 4A and 4B;

FIGS. 24A and 24B are schematic representations of the configuration of the RFIC shown in FIG. 23;

FIG. 25 is a block diagram of the acquisition chain of the RFIC shown in FIG. 23;

FIG. 26 is a graphical representation of data collection by way of the sensor assembly shown in FIGS. 4A and 4B;

FIG. 27 is a block diagram of the blood sensor assembly shown in FIGS. 4A and 4B, and corresponding transceiver;

FIG. 28 is a circuit diagram of the sensor assembly shown in FIGS. 4A and 4B;

FIG. 29 is a graphical representation of sensor assembly response to linear change in resistance;

FIG. 30 is a top plan view of the sensor assembly shown in FIGS. 4A and 4B;

FIGS. 31A and 31B are top plan views of the sensor assembly shown in FIGS. 4A and 4B during assembly;

FIG. 32 is a schematic representation of the procedure of FPS application using a stencil;

FIG. 33 is a top plan view of the sensor assembly shown in FIGS. 4A and 4B;

FIG. 34 is a schematic representation of the sensor assembly shown in FIGS. 4A and 4B during assembly;

FIGS. 35A and 35B are top plan views of the sensor assembly shown in FIGS. 4A and 4B with the DHA assembly in the flat and curled configurations, respectively;

FIGS. 36A and 36B are graphical representations of raw data received from the sensor assembly shown in FIGS. 4A and 4B, over a 60 second period;

FIG. 37 is a graphical representation of waveform amplitude distribution as a function of flow rate; and

FIG. 38 is a graphical representation of peak amplitude distribution of a submerged graft.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “a loop” can refer to one or more of such loops, and so forth.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

The word “or” as used herein means any one member of a particular list and, unless context dictates otherwise, can also include any combination of members of that list.

In the following description and claims, wherever the word “comprise” or “include” is used, it is understood that the words “comprise” and “include” can optionally be replaced with the words “consists essentially of” or “consists of” to form another embodiment.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

As further explained below, various conditions would benefit from real-time monitoring of disease progression. In particular, synthetic vascular grafts, vessel transplants, and systemic diseases would benefit from real-time monitoring.

### Synthetic Vascular Grafts

Prosthetic vascular grafts are oftentimes used for hemodialysis vascular access or in bypass surgery, and over one million grafts are implanted annually in the US. However, only 22% of implanted grafts remain free of complication in the first three years post-surgery. Monitoring the flow of blood through grafts can estimate if a clot or other type of graft-failure has occurred. Vascular grafts most commonly fail when the lumen diameter is reduced as endothelial cells migrate to the graft surface, known as intimal hyperplasia. When hyperplasia occurs, blood flow is reduced, pressure gradients required for homeostasis are lost, and the risk of blood clot is increased. In clinical terms, the vascular narrowing-stenosis-caused by intimal hyperplasia increases the likelihood that an embolus (blood clot) is trapped, triggering further clotting in stationary blood (thrombosis). If stenosis is not detected quickly and thrombosis results, most often the graft is not salvageable via surgery.

Due to the rapid time course of stenosis to thrombosis, which is difficult to detect given the shortcoming of imaging surveillance, 20 to 38% of vascular grafts fail in the first year after implantation, leading to hospitalization and other major health complications. A real-time detection of a 25% drop in blood flow could identify patients for early intervention to prevent complete graft loss. Therefore, the development of a simple, at-home blood-flow monitoring device could provide real-time detection of decreased flow and identify patients at risk for thrombosis. Previous approaches to measuring blood pressure or flow have been accomplished with sensors placed in the lumen of the graft, but risk accelerating graft failure by altering the mechanical structure of the graft.

### Vessel Transplants

More than 60,000 patients undergo coronary artery bypass grafting annually, costing $2.7 billion in the US as the population ages this number will continue to grow. Patients with critical limb ischemia and traumatic vascular repair surgeries oftentimes require revascularization. Additionally, patients recovering from cancer or trauma are now also receiving vascular reconstruction. In these situations, detection of a failed anastomosis is not always identified in time, making the monitoring of blood flow through the new anastomosis crucial. Early detection can enable minimally-invasive, prophylactic treatment such as percutaneous transluminal angioplasty to restore blood flow to prevent transplant loss.

### Systemic Diseases

Individuals with systemic blood pressure-related diseases are at-risk of a multitude of other conditions. Having a state of persistently low blood pressure in the arterial system is known as hypotension, while an extended period of high blood pressure is known as hypertension. Long-term hypertension is often undocumented due to its early-stage asymptomatic nature and lack of monitoring. Hypertension, the more common of the two, can lead to other medical complication such as strokes, cardiovascular diseases, renal failure, coronary artery disease, hypertensive heart disease and aortic aneurysms. Pressure in different organs or vessels is generally regulated, and a change in value over time may suggest an underlying disease progression or general patient health issues. Implantable blood pressure sensors can enable real-time monitoring of blood pressure (which varies throughout the day based on activity level, diet, and other factors), or inform new treatments such as drug dose or neuromodulation adjustment.

### Necessity for Monitoring Devices

Additionally, information through surveillance methods offers only a restricted snapshot of the flow or pressure within the vessel of interest. It is difficult to capture the trend in each person's disease progression, which can be useful in indicating in a timely manner the essential data for suitable intervention strategies. One solution to providing the accuracy of surveillance with the frequency of monitoring is through the development of implantable sensors. Technologically advanced sensors may provide accurate, easy-to-use, continuous pressure monitoring. These sensors can be used outside of clinical settings for at-home monitoring.

Previously developed devices for blood pressure monitoring include a magnetic flow meter attached to the aortic valve, a wired or battery-powered silicon cuff that uses ultrasonic Doppler shift to detect flow, and an ASIC-based inductively powered silicon nanowire sensor. These devices, however, are limited to the aortic valve location, require the use of a battery or wire, or need to be placed inside the graft to function, respectively. Further developments include a sensor that is partially embedded into the femoral artery, and a biodegradable flexible arterial-pulse sensor. The first device requires the puncturing of the vessel for insertion, while the second device dissolves after some time into the body, making it unfeasible for long-term monitoring.

One lifetime limiting element in implantable devices is the battery, which can cause infection or disease if it fails early and requires replacement. Furthermore, the encapsulation and size of the battery can limit the shape, function, or flexibility of the device. Devices can ideally, last in the body much longer without the failure mode of a primary cell battery. Additionally, batteries are often the largest components in a device and removing them may allow for further miniaturization. Therefore, for longevity of the implant and safety of the patient, implants without batteries are beneficial.

Wireless power transfer (WPT) is a promising methodology that may eliminate the need for batteries or wired connections, and provides further comfort and ease to the patient. Medical devices using WPT are approved by the FDA and in use today, unlike most other power transfer methodologies, such as ultrasound. WPT to miniaturized implants is challenging because the amount of power transferred is limited by the amount of energy the antenna can capture, and by the efficiency of the power harvesting circuit. Both cases present miniaturization challenges, although power harvesting circuits can be more readily miniaturized than flux-capturing inductive antennas. Further, misalignment between transmitting and receiving antennas reduces flux capture, which introduces multiple challenges in designing an implantable antenna fora graft or blood vessel.

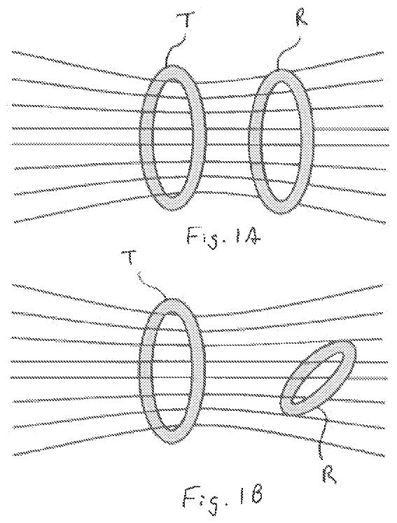

As shown in FIG. 1A, maximum power transfer occurs when two coils of the similar size are placed in close proximity and parallel, such that the secondary coil (R) captures much of the electromagnetic flux produced by the primary coil (T). However, as shown in FIG. 1B, when the secondary coil (R) is smaller, misaligned, or further away, far less flux is comparably captured by the second coil. The amount of magnetic flux is proportional to distance and alignment approximately by a 10% loss/mm and 2 to 4% loss/degree.

The problem with misalignment and coil size is especially significant when used as the receiving coil in a power harvesting circuit with an implant. The coil is necessarily small so as to fit within the implant, but results in comparably less energy captured. Additionally, because the antenna is within the patient, it cannot always be placed in the ideal location for receiving maximum energy from the Tx coil, and may even have an unknown orientation relative to the skin surface. The coil works best at a specific angle and falls off when not properly aligned.

Blood vessels or grafts, which are “tubular” organs, do not contain flat surfaces ideal for circuitry, and are not always of a known diameter. Traditional antennas, which are rigid, or flat, are not suitable for these uses. Further, it is desirable to place the transceiver as close as possible to the device for maximal WPT efficiency. Because vessels typically run parallel to the skin surface, this is often orthogonal to the axis of the vessel or graft. As such, the optimal direction of range for the antenna should be perpendicular to the vessel as shown in FIG. 2.

### Implantable Blood-Flow Sensor Assemblies

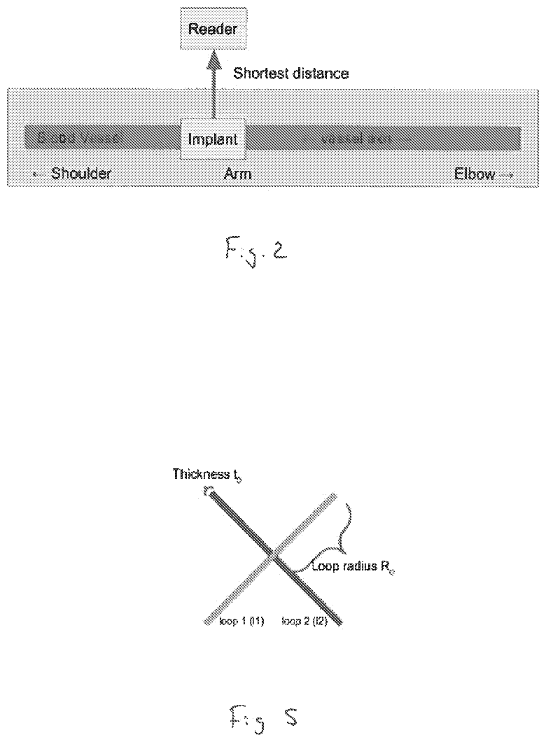

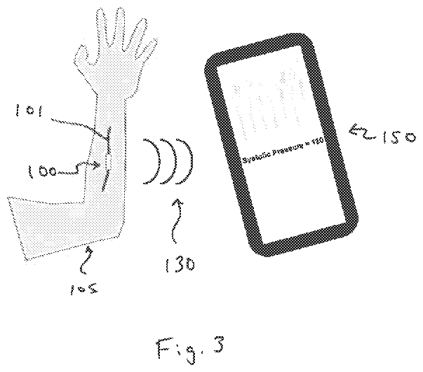

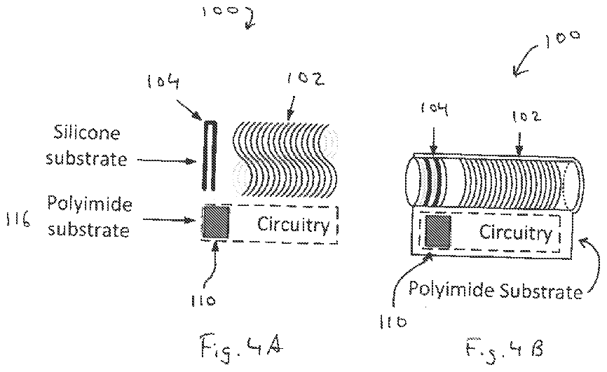

Referring now to FIGS. 3, 4A, and 4B, embodiments of implantable blood-flow sensor assemblies for monitoring blood flow within a tubular structure within a body of a subject are described in accordance with the present disclosure. For example, an embodiment of a blood flow sensor assembly 100 can include an antenna assembly 102 and a flexible sensor assembly 104 forming a cuff 103 that is configured to be positioned around the tubular structure 101 within the body 105 of the subject. As well, the blood flow sensor assembly 100 can include an integrated circuit 110 that is operably connected to both the antenna assembly 102 and the flexible sensor assembly 104. The blood-flow sensor assembly 100 uses wireless power transfer (WPT) 130 for communication with an external transceiver 150 and for powering implanted electronics. Preferably, the WPT system can use a split-double helix antenna (DHA) assembly 102, thereby enabling the formation of a cuff 103 that can be slipped around a tubular structure 101 in the body, such as an artery, a vein, a stent, etc. However, alternate embodiments may include antenna configurations other than DHAs. For example, antenna configurations that allow the antenna to be configured as a cylindrical cuff during operation fall within the scope of the present disclosure. The split DHA assembly 102 can wrap around vessels 101 (e.g., having a diameter of about 3 mm to about 8 mm) and can couple to a strain-sensitive flexible pulsation sensor (FPS) assembly 104, which can optionally comprise a carbon black-silicone nanocomposite. A radiofrequency integrated circuit 110 is operably connected to both the WPT antenna 102 and FPS assembly 104. The changes in strain encountered by the FPS assembly 104 during pulsatile flow can be measured and wirelessly transmitted to the transceiver 150, thereby enabling flow rate and blood pressure monitoring. This capability has been demonstrated on a vascular phantom. Exemplary configurations of each element of the various embodiments of the disclosed blood-flow assemblies 100 are discussed in greater detail below. In particular, the following disclosure includes separate sections directed exemplary, non-limiting features of the DHA Assembly, the Transceiver System, the Device Circuitry, the Flexible Pulsation Sensor (FPS), Stencil Fabrication, and Final Device Encapsulation.

### The Double-Helix Antenna (DHA) Assembly

Turning now to the development of the present disclosure, a set of equations that can be used to model the DHA and DHA-transceiver system are presented. Specifically, equations for DHA self-inductance and DHA-transceiver mutual inductance are utilized in optimizing the DHA for max power transfer given a specific size constraint. In a wireless power transfer (WPT) system, the efficiency of the system is directly proportional to the coupling coefficient. Specifically, the power transfer efficiency (PTE) between a receiver (RX) and transceiver (TX) is proportional to k:

$$η = \frac{{P}_{out}}{{P}_{in}} = \frac{Re [ {V}_{RX} {I}_{RX}^{*} ]}{Re [ {V}_{TX} {I}_{TX}^{*} ]} = \frac{{k}^{2} {ω}^{2} {L}_{TX} {L}_{RX} {R}_{L}}{{{R}_{P 1} ( {R}_{P 2} + {R}_{L} )}^{2} + {k}^{2} {ω}^{2} {L}_{TX} {L}_{RX} ( {R}_{P 2} + {R}_{L} )} , \qquad ( 1 )$$

where ω is the operating frequency, L_{TX} and L_{RX} the self-inductances of the two coils, R_{P1} and R_{P2} the parasitic resistance of the two coils, and R_{L} the resistance of the load. Generally, for a WPT system the coupling coefficient to an external transceiver is maximized for optimum efficiency, while considering constraints on antenna form factor.

Coupling coefficient optimization is achieved by modeling the DHA-transceiver coupling to avoid unnecessary prototype fabrication and characterization. The coupling coefficient between a DHA and an external transceiver coil (TX) is calculated as:

$$k = \frac{{M}_{DH / TX}}{\sqrt{{L}_{DH} {L}_{TX}}} , \qquad ( 2 )$$

where M_{DH/TX} is the mutual inductance between the DHA and transmitter, and L_{DH} and L_{TX} are the corresponding self-inductances. Each variable in this equation is calculated independently below.

To simplify the model, the DHA can be approximated as a series of intersecting circular loop antennas. Each pair of antennas is treated as a cell, and superposition is used to calculate the inductance of each cell separately, given a variable distance from each cell (winding pair) to the external transceiver. The following sections provide examples of how self- and mutual-inductance for each cell are modeled.

### Self-Inductance of DH Cell, L_{CELL}

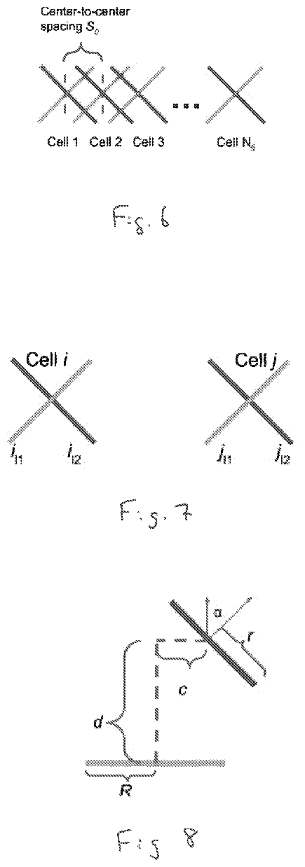

Each cell can be modeled as a pair of windings 11 and 12 that share the same center and bisect each other at 90 degrees (FIG. 5). The DHA has a total of N₀ cells, each having center-to-center spacing S₀ to the nearest cell (FIG. 6). The self-inductance of the DHA consists of the independent self-inductance of each cell, added with the mutual inductance of each loop to every other loop, i.e.,

$${L}_{DH} = {N}_{0} {L}_{CELL} + {\sum }_{i = 1}^{{N}_{0} - 1} {\sum }_{j = i + 1}^{{N}_{0}} {M}_{il 1 / jl 2} + {M}_{il 1 / jl 2} + {M}_{il 2 / jl 1} + {M}_{il 2 / jl 2} . \qquad ( 3 )$$

First, the self-inductance of an individual cell, L_{CELL} can be defined. The inductance of a circular loop with radius R and thickness t is approximated as:

$${L}_{LOOP} = {μ}_{0} R ( ln ( \frac{8 R}{t / 2} - 2 ) ) , \qquad ( 4 )$$

where μ₀ is the permeability of free space, R is the radius of the loop, and t the thickness of the wire.

The inductance of a cell is calculated as the self-inductance of each loop in addition to the mutual inductance between both loops. Since the loops are perpendicular, it is assumed that they do not share magnetic flux. Therefore, the mutual inductance between two loops that share a center and make up a cell is ignored, as represented by the following formula:

$${L}_{CELL} = 2 {L}_{LOOP} + {M}_{l 1 / l 2} = 2 {L}_{LOOP} + 0 = 2 {μ}_{0} R ( ln ( \frac{8 R}{t / 2} - 2 ) ) . \qquad ( 5 )$$

Inductance Between Cells, M_{l1/l2} and M_{l1/l2}

The inductance of the whole coil can be computed as the summation of self-inductance of all cells, in addition to the summation of the mutual inductance of every loop with every other loop. Although within each cell, it can be assumed there is no mutual inductance between orthogonal windings, this assumption does not hold between cells because each cell is separated by a nominal distance. Suppose there are N₀ cells (and therefore 2N₀ individual loops). Between two cells i and j, the inductance of two parallel loops, iₗ₁/jₗ₁ and iₗ₂/jₗ₂ will be equal (FIG. 7). Furthermore, perpendicular loops iₗ₁/jₗ₂ and iₗ₂/jₗ₂ will be equal. Therefore, equation 2 can be further simplified as:

$${L}_{DH} = {N}_{0} {L}_{CELL} + 2 {\sum }_{i = 1}^{{N}_{0} - 1} {\sum }_{j = i + 1}^{{N}_{0}} {M}_{il 1 / jl 1} + {M}_{il 1 / jl 2} . \qquad ( 6 )$$

To calculate the mutual inductance between two purely laterally misaligned loops M_{il1/jl2} and two laterally and angularly misaligned coils M_{il1/jl2}, the equation for two circular misaligned loops is used.

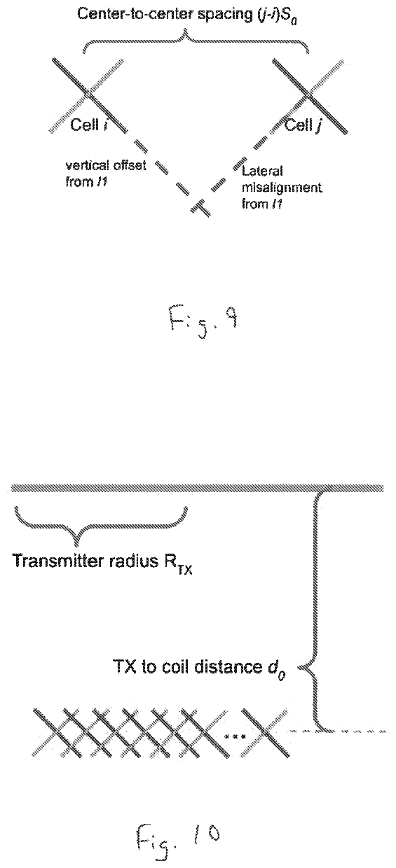

Some have sought to approximate the mutual inductance between two misaligned loops, M₁ and M₂ as in FIG. 8, both with basis from the Neumann formula:

$$M = \frac{{N}_{TX} {N}_{RX} {μ}_{0}}{4 π} ∮ ∮ \frac{{dl}_{TX} * {dl}_{RX}}{R} . \qquad ( 7 )$$

The distance between coil centers was first calculated, and then an initial equation for two coils without angular misalignment was defined parametrically. A Cartesian coordinate system was built around the primary coil, along with a second coordinate system around the secondary coil, with the two coordinate systems sharing a parallel x-axes and misaligned z-axes. A rotation matrix was then applied to transform between the coordinate systems, which allowed for the definition of the secondary coil with respect to the primary coil's center and coordinate system. dl_{Tx} and dl_{Rx} where then defined and plugged into the Neumann equation, then divided by the distance from coil to coil, resulting in:

$${M}_{1} = \frac{{u}_{0}}{4 π} {\int }_{0}^{2 π} {\int }_{0}^{2 π} \frac{Rr ( sin ( θ ) sin ( ϕ ) + cos ( α ) cos ( θ ) cos ( ϕ ) )}{\sqrt{\begin{matrix}{( R cos ( θ ) - r cos ( ϕ ) )}^{2} + \\ {( R sin ( θ ) - r sin ( ϕ ) cos ( α ) - c )}^{2} + ( r sin ( ϕ ) sin ( α )\end{matrix}}} \text{?} \qquad ( 8 )$$

$$\text{?} \text{indicates text missing or illegible when filed}$$

An alternative approach, from was also developed in a similar manner resulting in an identical numerator, but defined the coil-to-coil distance through another method, resulting in:

$${M}_{2} = \frac{{u}_{0}}{4 π} {\int }_{0}^{2 π} {\int }_{0}^{2 π} \frac{Rr ( sin ( θ ) s \dot{m} ( ϕ ) + cos ( θ ) cos ( ϕ ) cos ( α ) )}{\sqrt{\begin{matrix}( {R}^{2} + {r}^{2} + {c}^{2} + {d}^{2} - 2 Rc sin ( θ ) - \\ 2 Rr cos ( θ ) cos ( ϕ ) + ( 2 rc sin ( ϕ ) - \\ 2 Rr sin ( θ ) sin ( ϕ ) ) cos\end{matrix}}} \qquad ( 9 )$$

Where R is the radius of the first loop, r is the radius of the misaligned loop, and d is the perpendicular distance from the first loop to the parallel plane that the second loop's center lies in. Additionally, α is the angle of misalignment, and c is the lateral misalignment between the loops, as described in FIGS. 8 and 9.
In the case of the DHA, the vertical offset and lateral misalignment between cells i and j were equal as the loops are orientated at 45 degrees.

$$c = d = \frac{( j - i ) {S}_{0}}{√ 2} \qquad ( 10 )$$

For parallel loops (e.g., iₗ₁/jₗ₁ and iₗ₂/jₗ₂), the angle of misalignment, α, was 0. For perpendicular loops, (iₗ₁/jₗ₂ and iₗ₂/jₗ₁), α was π/2. The previous equations were then simplified to obtain a final calculation for L_{DH} as:

$${{{L}_{DH} = {N}_{0} {L}_{CELL} + 2 {\sum }_{i = 1}^{{N}_{0} - 1} {\sum }_{j = i + 1}^{{N}_{0}} [ {M}_{il 1 / jl 1} \vert}_{c = d = \frac{( j - i ) {s}_{0}}{\sqrt{2}} , α = 0} ] + [ {M}_{il 1 / jl 2} \vert}_{c = d = \frac{( j - i ) {s}_{0}}{\sqrt{2}} , α = \frac{π}{2}} ] . \qquad ( 11 )$$

### Mutual Inductance of DHA and Transmitter, M_{DH/TX}

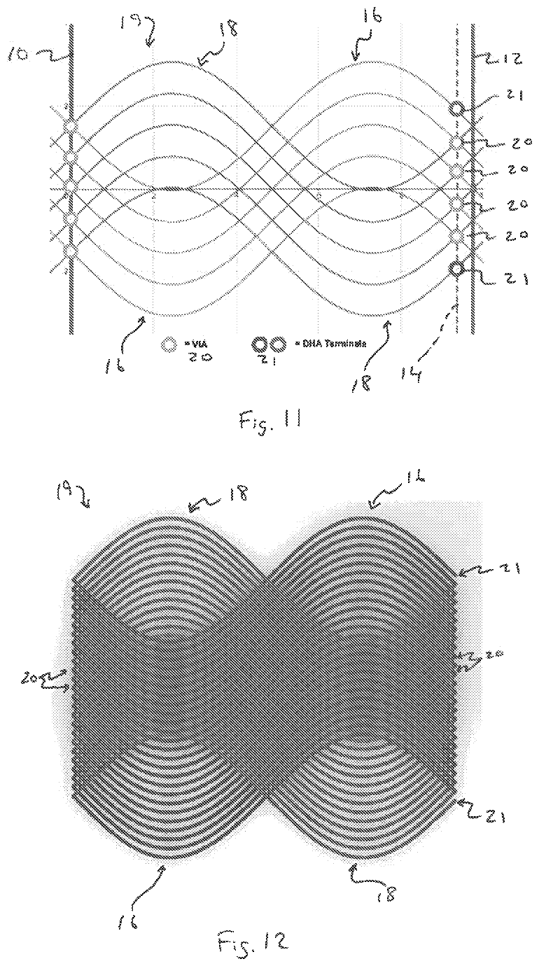

The transceiver coil is assumed to be a circular loop with several turns of the same radius (FIG. 10). In a similar fashion, the geometry is modeled as two misaligned loops with DHA/TX center-to-center distance d=d₀ and α=π/4. N_{TX} is defined as the number of turns in the transmitter coil.

The mutual inductance M_{DH/TX} between the DH coil and transmitter coil is calculated as:

$${M}_{DH / TX} = {N}_{TX} * 2 {{\sum }^{\frac{{N}_{0} - 1}{2}}}_{i = - \frac{{N}_{0} - 1}{2}} M {\vert}_{α = \frac{π}{4} , d = {d}_{0} , c = \vert i \vert {S}_{0}} . \qquad ( 12 )$$

For simplification, DHAs with odd number cells were analyzed and fabricated such that the limits on the summation were integers.

### Self-Inductance of Transmitter Coil, L_{TX}

Finally, the self-inductance of the transmitter coil is defined as:

$${L}_{TX} = {N}_{TX}^{2} * {L}_{LOOP} . \qquad ( 13 )$$

Using Equations 11, 12, and 13, the inductance of the DHA, inductance of the transmitter, and mutual inductance between the two were computed to estimate the coupling coefficient for antenna optimization.

The process and constraints for fabricating DHAs are now discussed. Constraints included manufacturer limitations, skin effect, proximity effect and the physical assembly of DHAs. The disclosed DHAs are designed to be compatible with standard flexible printed circuit board (PCB) fabrication, i.e. the DHA geometry is defined by drawn curves which are cast in copper after PCB production.

### DHA Trace Generation

The DHA is defined as a series of evenly spaced sine waves, cut off at the intersection of each wave's neighbor, as shown in FIG. 11. A via is placed at each intersection, allowing connection to the next cell of the DHA structure. Preferably, DHA traces are defined in SOLIDWORKS, then transferred to Altium Designer where vias and tuning capacitors are added before PCB fabrication.

### Creating DHA Trace Shape

A DHA with five cells (N₀=5) is modelled in FIG. 11. The lines 10 and 12 illustrate a full period of each sine wave, while the left line to the dotted line 14 illustrates the extent of the DHA. Intersections 20 of the first set 16 and the second set 18 of sine waves along the dotted line are the locations of vias 20. Vias 20 must be on the dotted line 14 rather than right-side line 12 to ensure a continuous path from one DHA terminal 21 to the other. Placing the vias 20 on the right-side line 12 would create multiple small non-continuous traces. Assigning S as the spacing between traces, and D as the diameter of the DHA, the sine wave modelling a single DHA top-layer “trace” as a function of horizontal location, x, is defined as:

$$\frac{D}{2} sin ( \frac{2 x}{D} ) , 0 \leq x \leq π D - ( \frac{D}{2} arcsin ( \frac{S}{D} ) ) . \qquad ( 14 )$$

For a larger N₀, traces are added by offsetting Equation 14 with multiples of S. To create the bottom-layer traces, the equations defining top-layer traces are multiplied by −1.

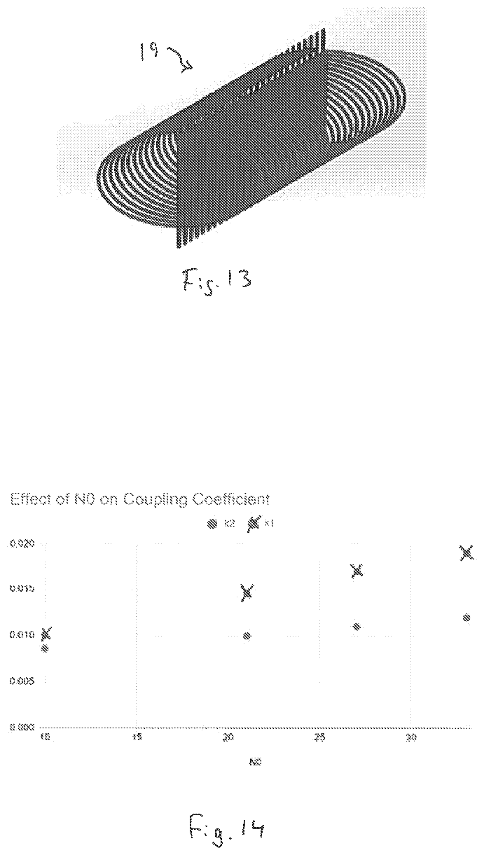

Variables D and S are defined as “global variables” in SOLIDWORKS, such that DHAs of various spacings and diameters can be dynamically generated. Using the “Equation Driven Curve Tool” a single DHA trace 19 is defined by Equation 14 and given an arbitrary thickness t. Then, traces are duplicated N₀ times with trace edge-to-edge spacing S₀ creating the series of top-layer traces 16, as shown in FIG. 12. These traces are then mirrored across the horizontal center to create the bottom-side traces 18. Using Solid Works “Flex” tool, a model of the wrapped DHA is generated, as shown in FIG. 13, to demonstrate how after bending, the flat DHA structure forms two interleaved magnetic windings.

After the DHA was defined in SOLIDWORKS, specific values for S, N, t, and D were chosen to generate physical prototypes with sizes to fit around blood vessels, within common manufacturer constraints. Initial revisions of the DHA were designed to fit snugly around 3.0-10.0 mm sized blood vessels, and be robust enough for final hand assembly. Therefore, parameters were limited by manufacturer constraints of mass production of flexible PCBs, as shown in Table 1.

TABLE 1
Manufacturer constraint and chosen parameters for all DHAs

| | Manufacturer | Chosen | Driven |
|---|---|---|---|
| Parameter | Constraint Value | Value | Value |
| Via minimum hole inner | 0.15 mm = 6 mil | 6 mil | — |
| diameter | | | |
| Via minimum annular ring | 6 mil | 6 mil | — |
| width | | | |
| Via minimum hole outer | 0.35 mm = 14 mil | — | 18 mil |
| diameter | | | |
| Track minimum width | 6 mil | — | — |

The first DHAs were manufactured with a vertical offset spacing between traces of S₀=15 mil and S₀=20 mil to test the effects of the proximity effect due to the compact size of the DHA, as shown in Table 2.

TABLE 2
Effect of spacing S0 on via and trace layout

| | | Driven | Driven |
|---|---|---|---|
| | Manufacturer | Value with | Value with |
| | Constraint | S₀ = 15 | S₀ = 20 |
| Parameter | Value | mil Chosen | mil Chosen |
| Via minimum hole | 11 mil | 17 mil | 22 mil |
| edge-to-edge spacing | | | |
| Track edge-to-edge | 6 mil | appx | appx |
| minimum spacing | | 8.3 mil | 11.7 mil |
| Via annular ring edge to | — | 5 mil | 10 mil |
| annular ring edge spacing | | | |

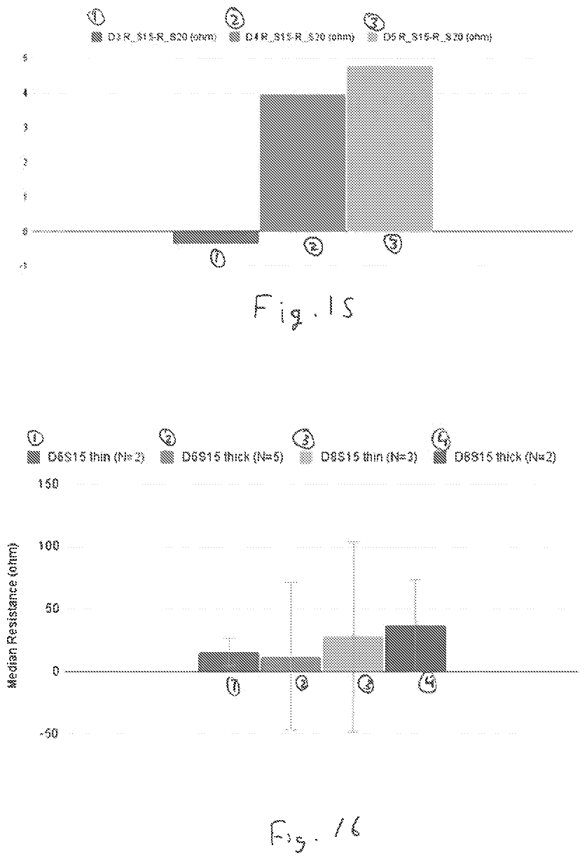

The effect of N₀ on the coupling coefficient is modeled mathematically as described above. A simulated DHA of diameter D₀=3 mm, spacing S₀=20 mil, D₀=6 mm was offset 5 cm from a transmitter with R_{TX}=5 cm, N_{TX}=10. As the size of the DHA increases significantly with additional cells, the coupling coefficient increases with diminishing returns, therefore, a value of N₀=25 was chosen for all DHAs assembled (FIG. 14). Once the proximity effect of the DHA was determined, as described below, a second batch of DHAs were made. Table 3 outlines the parameters associated with each batch of DHA.

TABLE 3
Associated DHA-specific parameters
for manufactured DHA batches

| | | |
|---|---|---|
| Parameter | Batch 1 | Batch 2 |

| | | |
|---|---|---|
| Number of cells N₀ | 25 | 25 |

| | | | | |
|---|---|---|---|---|
| Spacing S₀ | 15, 20 | mil | 15 | mil |
| Diameter D₀ | 3, 4, 5 | mm | 6, 8, 10 | mm |

### Proximity Effect

In fabrication and characterization, different DHAs with diameters and spacings were constructed. Therefore, the shorthand of DXSY is used, where X corresponds to the diameter of the DHA in mm, and Y corresponds to the trace-edge to trace-edge spacing, in mils.

In Batch 1, DHAs of diameter 3, 4, and 5 mm with spacings of 15 and 20 mil were manufactured to test proximity effect. Manufactured DHAs were measured using an Agilent 3955A Vector Network Analyzer. A difference of up to 5 ohms was measured between the average resistance of D5S15 and D5S20, as shown in FIG. 15. As this is a relatively small resistance, it was concluded that setting the spacing at 15 mils for Batch 2 was acceptable. Therefore, bench characterization of DHAs of differing geometries indicated that proximity effect was negligible at trace widths 15 to 20 mil.

### Skin Effect

At the operational frequency of 13.56 MHz, discussed in greater detail below, the skin effect must be accounted for in choosing the thickness of the traces for the DHA. DHA Batch 2a and 2b were fabricated to test the skin effect impact on DHA performance. Batch 2a had a thicker FPC and copper trace while Batch 2b was thinner in trace and FPC thickness (Table 4).

TABLE 4
DHA copper and FPS thickness for batches 2a and 2b

| Parameter | Batch 2a Chosen Value | Batch 2b Chosen Value |
|---|---|---|
| Copper thickness | 1 oz/cm² Cu = 36 μm | 0.5 oz/cm² Cu = 18 μm |
| FPC thickness | 0.13 mm | 0.1 mm |

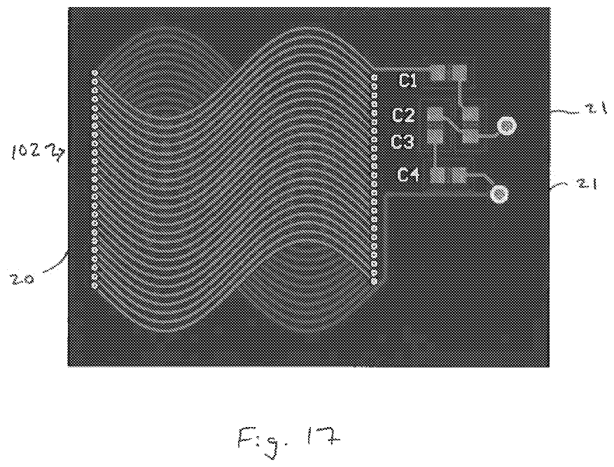

Because a small sample size was tested, statistical testing was not performed, but median resistances between DHAs did not vary beyond experimental variance. Therefore, it is concluded that the copper and FPC thickness did not have significant effect on the skin effect, as revealed in FIG. 16. Therefore, bench characterization of DHAs of differing geometries indicated that skin effect was negligible at trace thickness 18 to 36 μm.

### DHA Assembly

### Tuning Capacitors

DHA traces were generated in SOLIDWORKS then exported to Altium designer for PCB integration. To tune and reduce the reactive impedance of the DHA, separate tuning capacitors C1, C2, C3, and C4 were included on the PCB in series or parallel with each DHA. Inserting tuning capacitors either in parallel or in series with the DHA would decrease the reactance at a given frequency (FIG. 17). Regardless of if a series or parallel tuning capacitor was used, the capacitor value was selected to resonate with the DHA reluctance at a use frequency of 13.56 MHz, matching the drive frequency of common WPT systems.

The DHA was manufactured on a two-layer polyimide flexible printed circuit (FPC) with common fabrication parameters (Table 1, Table 2). A thinner FPC thickness was desired for easy rolling, but due to manufacturer constraints, only 0.5 oz copper was used on 0.1 mm FPC thickness. Due to the skin effect, the lower copper thickness had the potential to increase resistance. Therefore, Batch 2a and Batch 2b were fabricated to compare the difference in copper thickness, as mentioned earlier.

TABLE 5
Copper and FPC thickness for all DHA batches

| | Batch 1 | | Batch 2a | | Batch 2b | |
|---|---|---|---|---|---|---|
| | Chosen | | Chosen | | Chosen | |
| Parameter | Value | | Value | | Value | |
| Copper | 1 oz Cu = 35 μm | | 1 oz Cu | | 0.5 oz Cu = 18 μm | |
| thickness | | | | | | |
| FPC | 0.13 | mm | 0.13 | mm | 0.1 | mm |
| thickness | | | | | | |
| Diameter | 3, 4, 5 | mm | 6, 8, 10 | mm | 6, 8, 10 | mm |

| | | | | | |
|---|---|---|---|---|---|
| Spacing | 15 mil, 20 mil | 15 | mil | 15 | mil |

Once manufactured, the flat DHA was wrapped such that the column of vias 20 (FIG. 11) at one end overlapped those at the other end. For initial prototypes the DHA was wrapped with polyimide tape to retain its shape during characterization.

### Volume Estimation

The minimum length of the DHA is estimated as:

$${l}_{DHA} = {S}_{0} ( {N}_{0} - 1 ) + 2 ( \frac{{R}_{0}}{\sqrt{2}} + \frac{{w}_{0}}{2} ) , \qquad ( 15 )$$

where R₀ was the radius of the coil and w₀ was the width of the trace.
The cross-sectional area of the coil A_{DHA} was approximated as

$${A}_{DHA} = π {R}_{0} ( \frac{{R}_{0}}{\sqrt{2}} ) , \qquad ( 16 )$$

with a volume V_{DHA} as

$${V}_{DHA} = {l}_{DHA} {A}_{DHA} . \qquad ( 17 )$$

### Theoretical and Measured Self-Inductance

To verify the developed equations, DHAs with diameters of 3, 4 and 5 mm, with spacings of 15 and 20 mil were constructed and characterized. The self-inductance of each DHA coil was computed using both equations for misaligned coils mutual inductance, resulting in two theoretical values, L_{DH1} and L_{DH2}. The inductance of two sets of each DHA was then measured at 100 kHz (using Hioki LCR Meter IM3533), as shown in Table 6. As the calculations do not factor in parasitics that arise at higher frequencies, a relatively smaller frequency of 100 kHz was arbitrarily chosen.

TABLE 6
Measured and theoretical values for D3S15, D3S20, D4S15, and D4S20

| Name | D3S15 | D3S20 | D4S15 | D4S20 | D5S15 | D5S20 |
|---|---|---|---|---|---|---|
| Diameter (mm) | 3 | 3 | 4 | 4 | 5 | 5 |
| Spacing (mil) | 15 | 20 | 15 | 20 | 15 | 20 |
| Theoretical L_{DH1} (uH) | 0.75 | 0.69 | 1.25 | 1.10 | 1.85 | 1.61 |
| Theoretical L_{DH2} (uH) | 1.24 | 1.10 | 2.00 | 1.77 | 2.9 | 2.61 |
| L_{DH} Measured Set 1 (uH) | 0.82 | 0.73 | 1.37 | 1.21 | 2.13 | 1.89 |
| L_{DH} Measured Set 2 (uH) | 0.86 | 0.69 | 1.41 | 1.24 | 2.04 | 1.87 |

In the second batch of DHAs produced, the spacing was set to 15 mil as it was determined that the proximity effect did not significantly increase the DHA's resistance and resulted in a more compact DHA. Table 7 shows the theoretical range of values for each DHA along with the average measured inductance of n=3 fabricated DHAs for each diameter near the maximum measurable frequency of the instrumentation, 5 MHz.

TABLE 7
Measured and theoretical values for
D6S15, D8S15, and D8S15 at 5 MHz

| Name | D6S15 | D8S15 | D10S15 |
|---|---|---|---|
| Theoretical L_{DH1} (uH) | 2.55 | 4.30 | 6.43 |
| Theoretical L_{DH2} (uH) | 3.92 | 6.18 | 8.67 |
| L_{DH} Measured (uH) at 5 Mhz | 3.09 | 5.15 | 9.00 |

For DHAs of diameter 6 and 8 mm the developed calculations were accurate as they were between the calculated theoretical values. For DHA of diameter 10 mm the previous discussed assumptions resulted in an under-estimation of the true inductance by 3.7%.

TABLE 8
Measured and theoretical values for
D6S15, D8S15, and D8S15 at 5 MHz

| Name | D6S15 | D8S15 | D10S15 |
|---|---|---|---|
| Theoretical L_{DH1} (uH) | 2.55 | 4.30 | 6.43 |
| Theoretical L_{DH2} (uH) | 3.92 | 6.18 | 8.67 |
| L_{DH} Measured (uH) at 5 MHz | 3.09 | 5.15 | 9.00 |
| L_{DH} Measured (uH) at 13.56 MHz | 3.50 | 9.81 | −15.23 |

For the 6 and 8 mm diameter DHAs the parasitic capacitance increased the effective inductance of the DHA. For the larger 10 mm diameter DHA at 13.56 MHz the parasitic capacitance significantly increased, making the DHA function essentially as a capacitor at the desired frequency. For this reason, an 8 mm DHA was used as the communication antenna within the device used to conduct ex-vivo experiments. This size provided the largest functioning communication distance, small enough to still act as an inductor at the desired frequency, and still appropriately sized to for wrapping a blood vessel or graft.

### Mutual Inductance and Coupling Coefficient Verification

A circular transceiver coil of diameter 100 mm with N_{TX}=3 turns was constructed. Arbitrary frequencies of 100 kHz (for D4S15) and 500 kHZ (for D5S15) were chosen with DHAs and transmitter coils remaining untuned, as the mutual inductance between two coils should not be affected by tuning or frequency based on the previously discussed equations. The mutual inductance between two elements at a given separation distance was measured.

The DHA and transmitter were put in series and then both terminals were connected to the LCR meter in an “aiding configuration” such that the mutual inductance between the two coils added to the self-inductance of the DHA and Tx coil (FIG. 19).

$${L}_{aiding} = {L}_{DHA} + {L}_{TX} + 2 {M}_{DHA / TX} \qquad ( 18 )$$

Then, the terminals of the DHA were flipped to an opposing configuration such that the mutual inductance between the coils subtracted from the self-inductance of each coil.

$${L}_{opposing} = {L}_{DHA} + {L}_{TX} - 2 {M}_{DHA / TX} \qquad ( 19 )$$

Finally, the difference between the measured inductances was divided by 4 to achieve the mutual inductance at that given distance (FIG. 4-4).

$${M}_{DHA / TX} = \frac{{L}_{aiding} - {L}_{opposing}}{4} \qquad ( 20 )$$

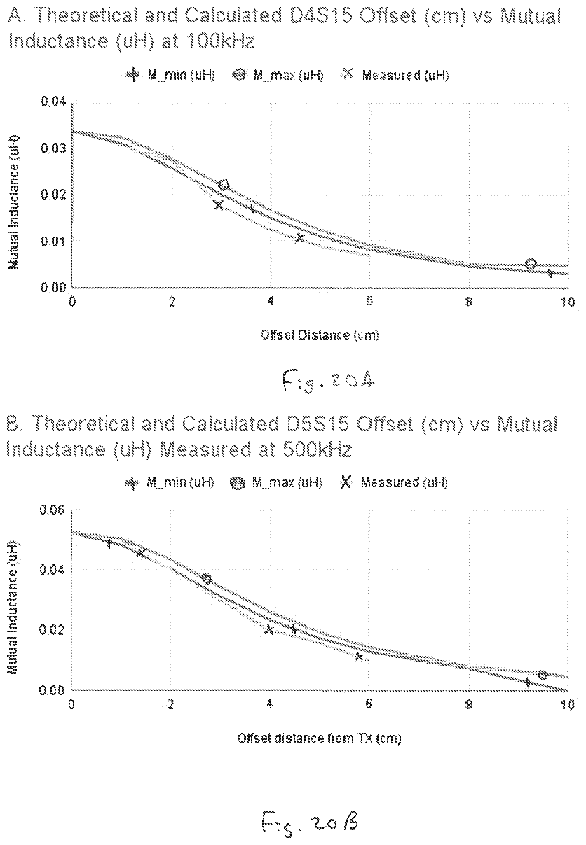

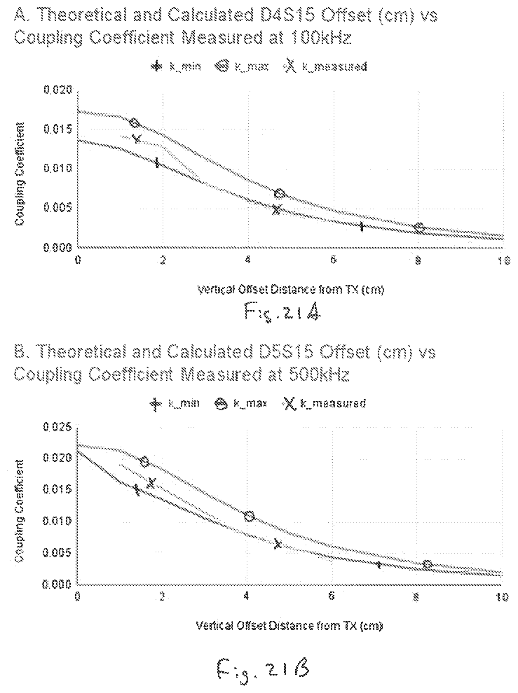

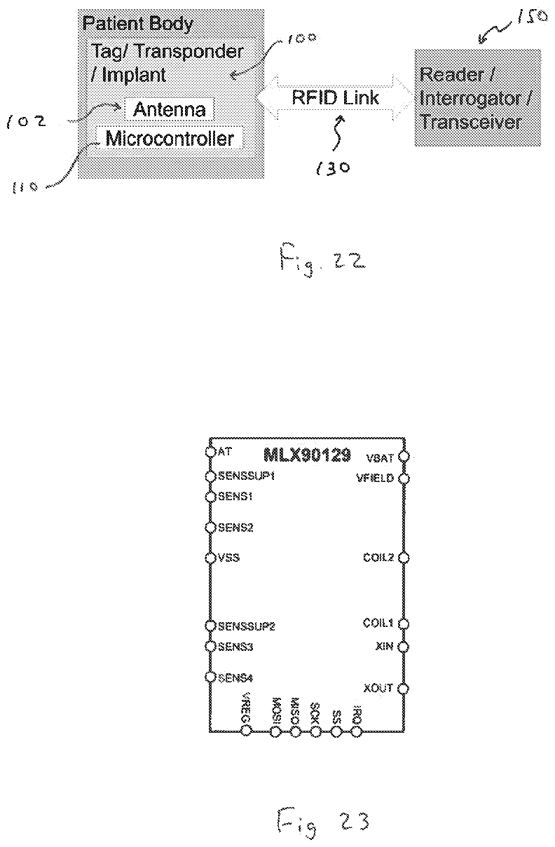

This process was repeated for offset distances of 1-6 cm. Beyond 6 cm the mutual inductance between the elements was too small to be measured. Similarly, using the two approximation equations for mutual inductance, theoretical min and max values for the mutual inductances were computed as shown in FIGS. 20A through 21B.

Of note is that for at a given offset distance, the mutual inductance changed as the DHA was translated within the plane. The equations assume that the point of maximum mutual inductance between the two elements was with the DHA located above the center of the transmitter. However, in measuring, the location of maximum mutual inductance was often directly above the wire edge of the transmitter. This is because the magnetic field from the transmitting coil is not uniform, a known issue for coils of this transmitter geometry (larger diameter, thin wire size, small length). This is something that can be further explored in future investigations. The DHA was translated within the offset plane and the measured mutual inductance was selected as the max mutual inductance found at each given offset. Then, using the mutual inductance, plots of the experimental vs measured coupling coefficient were generated.

For diameters of 3 to 8 mm, the measured inductance lied between the two bounds of the theoretical values previously discussed at lower frequencies (5 MHz and below). Unmodeled parasitic effects significantly changed DHA inductances at elevated frequencies (13.56 MHz) with 10, 58 and 336% error for D6S15, D8S15, and D10S15 respectively. While further work can accommodate parasitic effects into the mutual inductance models, these formulas are effective for initial DHA prototype development.

### Transceiver System

In order to create a fully wireless implantable device 100, the FPS assembly 104 and DHA assembly 102 include additional circuitry to communicate to an external transceiver 150 (FIG. 22) located outside of the body. To miniaturize the implanted system, wireless power transfer (WPT) is used instead of an internal battery. Therefore, the implanted circuitry for sensing and transmitting to the external transceiver is configured to harness power and communicate sensor measurements over the WPT field using a radiofrequency identification (RFID) protocol.

The “tag”, or “transponder”, is an RFID-capable unit that communicates with a “reader”, or transceiver 150 (FIG. 22). In this case, the implantable sensor assembly 100 acts as an RFID tag. RFID tags fall into one of 3 categories: active, semi-active, and passive. Active tags derive their power on-board using a battery which powers the circuitry for operating the device and communicating to the transceiver. Active tags can initiate communication to the reader, and the communication range can work up to hundreds of meters.

Semi-passive RFID tags contain a battery to extend the communication range but are not able to initiate communications. They are only active when queried by a reader. For example, an electronic tollbooth tag is not constantly transmitting information. Only when queried by the tollbooth, the on-board battery lets the tag be read from a considerable distance. These tags have a similar communication range to active tags and can get up to the hundreds of meters.

Passive tags do not contain any power sources. Through WPT, power is transmitted by the transceiver, used to operate the device (for example, measuring a sensor), and used to communicate data back to the transceiver. The absence of a battery reduces device failure modes and allows for extreme miniaturization. Passive tags generally operate at one of three frequencies: low frequency (LF) 120-140 kHZ, high frequency (HF) 13.56 MHz and ultra high frequency (UHF) 868-928 MHz. Generally, the higher the operational frequency the farther the communication range with LF and HF functioning up to 20 cm, and UHF up to 3 meters. The tag communicates to the transceiver through one of two techniques depending on the frequency. For LF and HF operation, the RFID tag-transceiver system operates in the near field and thus uses inductive coupling. For UHF operation, backscattering technique is used.

The implanted system is designed to operate within the HF 13.56 MHz industrial, scientific and medical (ISM) band, as it allows for further communication distance relative to LF, and has moderate data speeds. Since the tag will be potentially deep within human tissue, communication range of at least two centimeters is needed for the external transceiver to communicate with the implanted tag. Operation at UHF would impose limitations on RF exposure which reduce the maximum permissible WPT energy, reduce quality factor due to skin effect in the DHA, and limit system range due to the higher dielectric attenuation at UHF within human tissue. Higher frequencies result in higher eddy current losses and off-the-shelf passive tags are also less common, hindering prototyping.

Within the 13.56 MHz HF ISM band there are two RFID protocols. The first is ISO14443 which is used for proximity cards used in secure identification (e.g. keycards) and have relatively higher 106 kbps bit rate. The second standard, ISO15693 is the standard for vicinity cards that can comparatively be read at greater distances and require less power (smaller necessary magnetic field) but have relatively lower data transfer speeds of 26 kbps.

As reading distance is proportional to the flux capture area of the tag's inductive antenna, the need to miniaturize a medical implant implies worse RFID performance. Therefore the implanted system adopted the ISO15693 communication standard to operate at lower magnetic field strength. The use of this standard allows longer readout range, at the expense of data transfer speed.

### RFID Tag

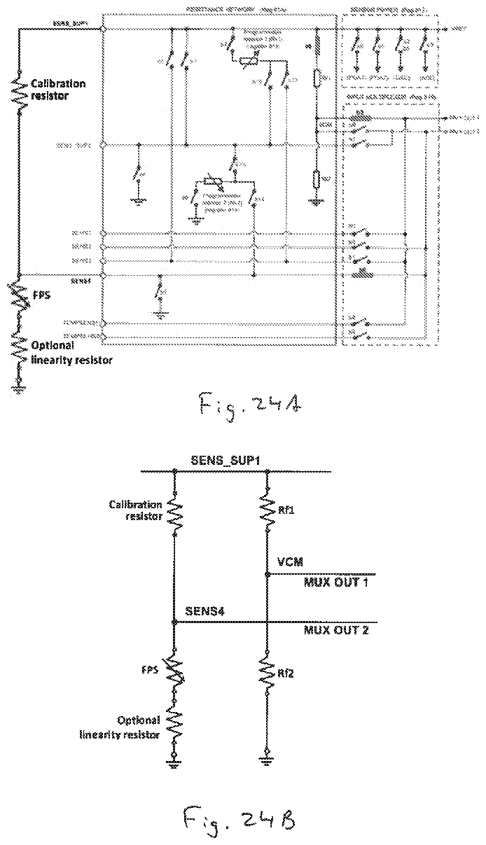

Two off-the-shelf ISO 15693 ICs supporting external sensor measurement using an ADC were identified: Texas Instruments RF430FRL152H and Melexis MLX90129. The Melexis MLX90129 (FIG. 23) was chosen as the RFIC to build the system around. However, it is contemplated that other suitable RFIC models and/or configurations can be used. The primary elements that were necessary to add for a fully functioning device were the FPS+calibration resistor, DHA, and power regulation circuitry, discussed in greater detail below.

### Transceiver Device and Code

Also adhering to ISO15693 standards, the NXP PN5180 was chosen as the transceiver chip for bench testing. This IC had an off-the-shelf module (PN5180 Card Reader Module) available with pre-existing software libraries, and a built-in 13.56 MHz tuned antenna. The NXP PN5180 module communicated to an Arduino Teensy 4.1 through SPI, enabling serial communication to a PC over a USB port for data readout from the RFID tag. An Arduino Teensy 4.1 was then connected (Table 9) to the ISO15693 capable transceiver chip through SPI.

TABLE 9
Teensy to NXP PN5180 Connection

| Teensy 4.1 Connection | NXP PN5180 Module Connection | |
|---|---|---|
| 5 V | +5 | V |
| 3 V 3 | +3.3 | V |

| | |
|---|---|
| Pin 3 | RST |
| Pin 4 | NSS |
| MOSI (Pin 11) | MOSI |
| MISO (Pin 12) | MISO |
| SCK (Pin 13) | SCK |
| Pin 5 | BUSY |
| GND | GND |

An existing library was utilized to communicate with the PN5180. Code was written to connect to an individual MLX90129, configure it with relevant sampling rate, ADC settings, internal resistor configuration, and PGA configuration. Then, a loop ran where the sensor tag was repeatedly polled to return samples as quickly as possible. With this reader, the maximum achievable sampling rate (12 Hz) was limited by the reader-tag communication delay.

Although exemplary transceiver configurations are disclosed herein, it is contemplated that other transceiver structures and configurations can be employed without departing from the goals of the present disclosure.

### MLX90129 Internal Configuration and Function

The MLX90129 resistance network was configured as shown in FIGS. 24A and 24B. A Wheatstone bridge was formed with the calibration resistor and FPS as one branch (outside the IC) and second branch consisting of Rf1 and Rf2 internal to the chip, with b6 closed. The input multiplexer was configured such that outputs of the bridge went to MUX OUT 1 with b3 and b6 closed.

### Data Acquisition

The differential output voltage of the Wheatstone bridge was amplified and digitized by the sensor interface signal chain, as shown in FIG. 25. Resistance changes in the FPS produced voltage differences in the bridge output; the differential voltage was amplified by the first programmable gain amplifier (PGA) 40. A digital-to-analog (DAC) converter 42 applies a programmable offset after the first gain stage. The digital offset was used to compensate when the calibration resistor and resting FPS resistance were not equal. This compensated differential voltage was further amplified by a second PGA 44 and then converted to a digital datastream code by the analog-to-digital converter (ADC) 46. The MLX90129 documentation supplies the following equation that relates the data stream code to the change in voltage of the sensor:

$$( \frac{{V}_{REF}}{{2}^{ENOB}} * {D}_{10} ) - \frac{{V}_{REF}}{2} = ( [ ( ( {GAIN}_{PGA 1} * Δ {V}_{sensor} ) + {N}_{10} * \frac{{V}_{REF}}{128} ] * {GAIN}_{PGA 2} . \qquad ( 21 )$$

This equation is then rearranged to solve for the change in voltage of the sensor given a data stream output:

$$Δ {V}_{sensor} = \frac{\frac{( \frac{{V}_{REF}}{{2}^{ENOB}} * {D}_{10} ) - \frac{{V}_{REF}}{2}}{{GAIN}_{PGA 2}} - {N}_{10} * \frac{{V}_{REF}}{128}}{{GAIN}_{PGA 1}} . \qquad ( 22 )$$

In experiments, as the FPS had significant resistance change when wrapped around the graft, the PGA gains were kept low to avoid saturation. With typical sensor values, nominal PGA gains in Equation (22) were programmed onto the MLX90129 and held constant over all experiments (Table 10). The N₁₀ offset values were adjusted as needed to account for differences in FPS and calibration resistor, such that the nominal output at zero strain was near the ADC midpoint.

TABLE 10
Values and definitions for MLX90129 data acquisition equation

| | |
|---|---|
| D₁₀ | Data stream outputted to serial port |
| ENOB | Effective number of bits, determined as either |
| | 8, 9, 10, or 11 depending on MLX90129 configuration |
| V_{REF} | Reference voltage of the MLX90129, (depends on low |
| | voltage option) appx 2.0~2.2 V |
| ΔV_{SENSOR} | Differential voltage from the sensor |
| N₁₀ | Decimal number programmed into the DAC, acts as offset |
| GAIN_{PGA1} | Gain of first PGA, set to 8 for all |
| | experiments (minimum value) |
| GAIN_{PGA2} | Gain of second PGA, set to 1 for all |
| | experiments (minimum value) |

### Data Collection

After the transceiver device configured the MLX90129 chip and polled it for data readings from the sensor, received readings were recorded via the serial port of a computer. A MATLAB program then read from the serial port and stored individual reading with associated timestamps for further analysis, as shown in FIG. 26. Optional functionality of real-time plotting of readings was created to examine flow.

For experiments the FPS sensor was wrapped around a (phantom) blood vessel and connected directly to the sensor terminals of the MLX90129, discussed in greater detail below, creating the system block diagram shown in FIG. 27.

### Device Circuitry

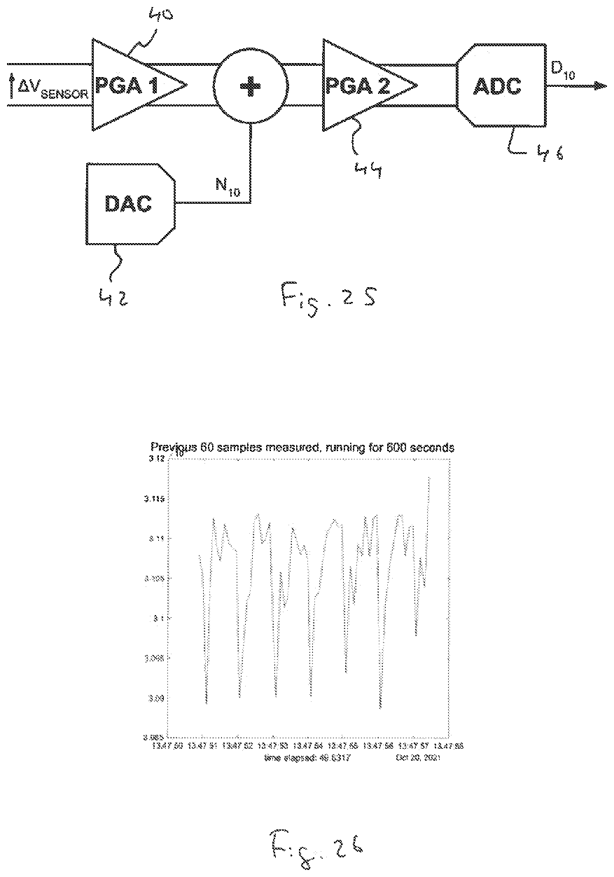

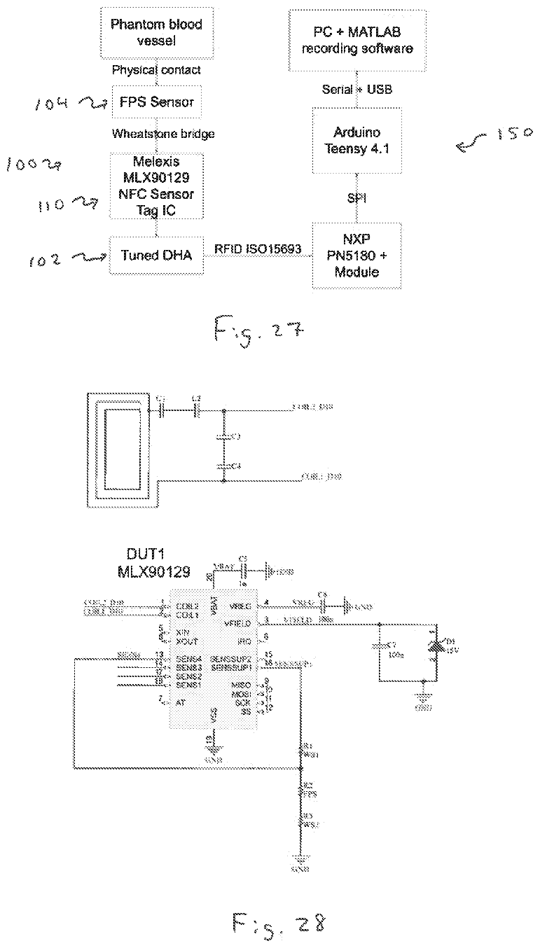

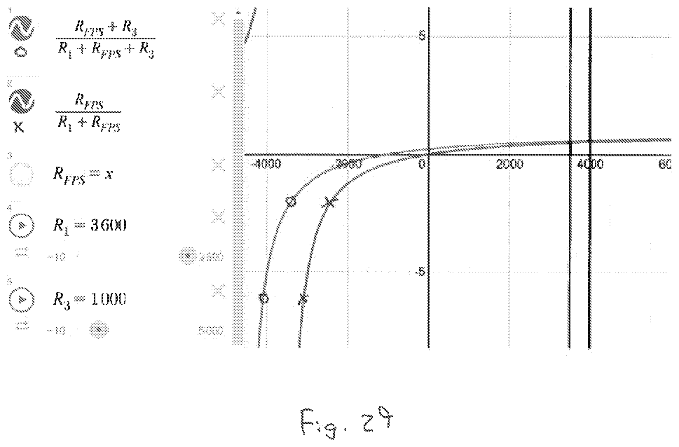

The sensor interface circuit was developed around the MLX90129 chip as shown in FIG. 28. R2 represents the FPS with variable resistance based on blood vessel strain. R1 is the calibration resistor on the external branch of the Wheatstone bridge, chosen to match the FPS resistance at zero strain. This resistance is determined after the FPS is patterned onto the substrate to ensure good matching for optimal sensing dynamic range. R3 is an additional optional resistor for linearity. The FPS is on a branch of the Wheatstone bridge acts as a voltage divider, such that the voltage measured (compared to the reference voltage as described above), doesn't change linearly as the resistance of the FPS changes linearly. FIG. 29 illustrates two vertical bars 50a and 50b that indicate the relevant bounds of a realistic FPS from unstretched (3.5 k ohm) to stretched (4 k ohm), as blood flows through the vessel it is wrapped around. Adding an R3 value brings the operational range further from the asymptote and thus to a more linear part of the plot. As the operational range was already in a relatively linear part of the plot, R3 was shorted in tests with the final device.

Multiple optional spots for tuning capacitors were added to allow for the creation of specific capacitance in the case of lacking capacitor value in the laboratory. Specifically, putting two capacitors in series would create an equivalent capacitance of

$${C}_{EQ} = \frac{1}{\frac{1}{{C}_{1}} + \frac{1}{{C}_{2}}} . \qquad ( 23 )$$

Tuning can be done in parallel or series but was chosen to be done in series for the initial embodiments. C1 and C2 are series tuning capacitors for the DHA, chosen to resonate with the DHA at 13.56 MHz, after accounting for additional capacitance introduced by the MLX90129 IC, and environmental capacitance when submerged in water. C3 and C4 are additional optional parallel tuning capacitors for the DHA that remained unused in the final device. C5 and C6 are filter capacitors used to eliminate ripple on the rectified output voltage and improve transient response. C7 and D1 form a shunt regulator to protect the MLX90129 from overvoltage, which may occur at short-range WPT operation.

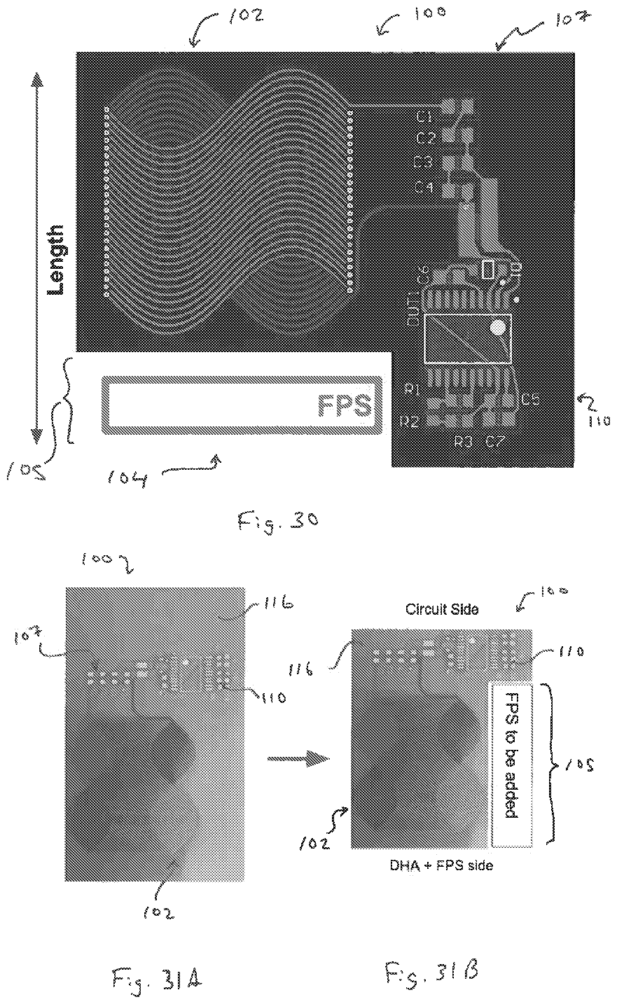

Referring now to FIG. 30, the flexible PCB layout was created using Altium Designer. Components such as the DHA assembly 102, tuning capacitors 107, and radiofrequency integrated circuit 110, are aligned between flexible base and top layers of polyimide substrate 116 so that the max length of the device is determined by the DHA assembly 102 and FPS assembly 104 aligned vertically. R2 acts as the terminals for the FPS which extends left. The polygon fills connecting the underside terminal of the DHA, and two COIL pads (FIG. 28) on the MLX90129 were created so that in testing the DHA could be cut and separated from the other circuitry, or directly probed and soldered for tuning and debugging.

### Circuitry Assembly and Packaging

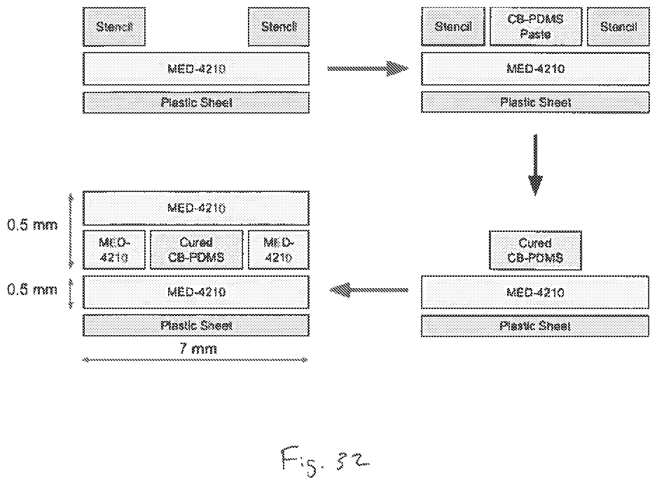

Referring now to FIGS. 31A and 31B, before assembling, a rectangle for the FPS is cut out of the flexible printed circuit (FPC) board. To make the device as compact as possible excess board material on the circuit-side was significantly trimmed, and a slight excess of material on the DHA+FPS side was left to assist with manual rolling of the DHA once assembled. The FPC was reflow soldered with all required components, including tuning capacitors for respective DHA diameter. Tuning capacitance added was determined as

$$f = \frac{1}{\sqrt{2 π LC}} = 13.56 MHz \to {C}_{tuning} = \frac{{( \frac{1}{13.56 \text{?} {10}^{6}} )}^{2}}{2 π {L}_{DHA}} , \qquad ( 24 )$$

$$\text{?} \text{indicates text missing or illegible when filed}$$

with L_{DHA} representing self-inductance of the DHA.

To continue assembly, a 0.5 mm thick layer of MED-4210 PDMS was manually cast on a plastic sheet. The FPC was placed on the uncured PDMS to encourage bonding. The MED-4210 silicone PDMS was then cured at 60° C. for 30 minutes, adhering the FPC to the PDMS.

### FPS Fabrication

The device can monitor real-time blood flow through a vascular graft by using a piezoresistive flexible pulsation sensor (FPS). Preferably, the sensing element is comprised of carbon-black nanoparticles suspended in polydimethylsiloxane (CB-PDMS), which produce a metal-free strain sensor with linear resistance-strain response. Because the FPS is based on a PDMS substrate, it exhibits large strain range, greatly exceeding maximum strain of conventional metal sensors, and exceeding the expected strain range of natural blood vessels and synthetic grafts.

The flexible pulsation sensor (FPS) is produced by creating a CB-PDMS paste, which is then patterned using a stencil. After curing, it is encapsulated in biocompatible PDMS to insulate the sensing element from conductive bio-media. It was determined that 14% CB suspension is optimal for a linear resistance-strain response. Component weights for the CB-PDMS were calculated to achieve a final mix of 14% by weight, as shown in Table 11.

TABLE 11
Ingredient list and amount for CB-PDMS fabrication

| Ingredient | Amount by mass |
|---|---|
| Sieved carbon black particles | 14% |
| Toluene | Enough to undersaturate CB in |
| | mixture, appx 20 g for 1 g of CB |
| Ecoflex 00-10 part A | 43% |
| Ecoflex 00-10 part B | 43% |

The following steps were followed in CB-PDMS preparation:
1. Carbon black particles were first grinded into a mortar and pestle
2. The particles were then sieved and allowed to fall onto a dish until the necessary amount of carbon black was obtained
3. The sieved CB was added to a glass vial with approximately enough toluene such that the mixture is undersaturated, and was sonicated for 30 min
   a. Important note: the relationship between CB to toluene required to undersaturate CB is not linear, 1 g of CB requires approximately 20 g of CB, whereas 0.5 g require approximately 15 g of toluene
4. Added Ecoflex 00-10 (Smooth-On) part A to the mixture and sonicated for 30 min, 20 s on 30 s off at 20% amplitude (Qsonica Q500 with 1.8″ probe tip)
5. Added Ecoflex 00-10 (Smooth-On) part B to the mixture and sonicated at 10 min at same settings as above
6. Using a stir bar and stirrer, the solution was mixed for two hours or until toluene was fully evaporated (FIG. 6-6)
   a. The solution was kept at room temperature to prevent PDMS curing
7. Using a stencil, the CB-PDMS paste was applied onto the cured MED 4210, which was pre-bonded to the assembled FPC as described previously (FIG. 32).
8. Allowed the CB-PDMS to cure for an hour at 60, and then removed stencil.
9. Finally, the exposed FPS and top side of the FPC (MLX90129 IC, circuitry, and DHA) was coated with MED-4210. PDMS was manually applied to maintain a thin layer (about 0.5 mm) above all circuits. To improve FPS elasticity a thinner layer of about 0.25 mm was applied directly above the FPS. Greater FPS elasticity is needed to ensure the FPS does not constrict the blood vessel; this also maximizes sensor strain and improves signal-to-noise ratio.

### Stencil Fabrication

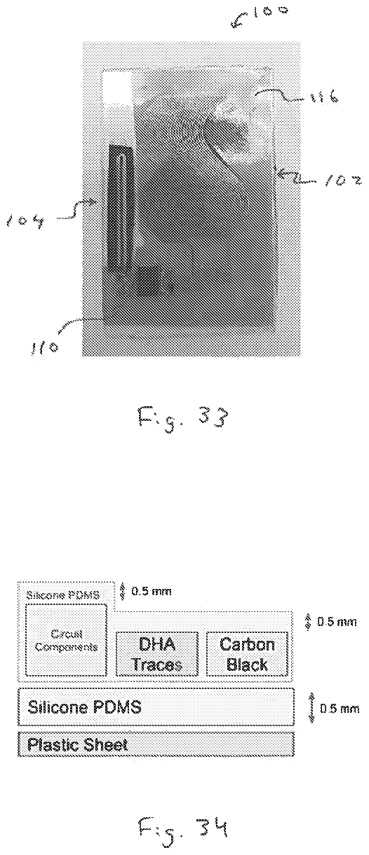

The CB-PDMS paste was patterned to the final FPS strain sensor dimensions using a stencil. Strain sensor dimensions directly affect sensitivity, thinner tracks and multiple parallel tracks lead to greater resistance changes for the same uniaxial strain. However, for large aspect ratios, this requires stencils with long, thin, cantilevered segments, which tend to deflect during patterning, or allow material underfill. To partially mitigate these issues, an adhesive stencil was used which was tacky enough to stick to the PDMS substrate, preventing member deflection. Stencils were cut out of matte vinyl using a Roland GX-24 vinyl cutter. The sticky back side of the stencil was removed and then applied onto the cured MED-4210, with edges of the stencil on top of the pads for the FPS, as shown in FIG. 33.

Stencil dimensions were chosen to enable patterning onto a surface-mount device 0603 layout, with pads 0.95×0.80 mm separated by 0.50 mm. 0603 pads were chosen as they were small to keep the device compact, but large enough to be manually assembled. In further work, 0603 pad-size can be further reduced. Four different types of stencils were tested, two different lengths, with shapes “U” and “W”. The “W”-shaped stencils made the device slightly wider and applying the CB-PDMS paste more difficult as it increased the chance for the paste to go under the stencil, so “U”-shaped stencils were used in the later embodiments. The longer 26 mm “U”-shaped stencil was ultimately used as it could be wrapped around the vessel up to two times, resulting in a greater sensitivity.

### Final Device Encapsulation

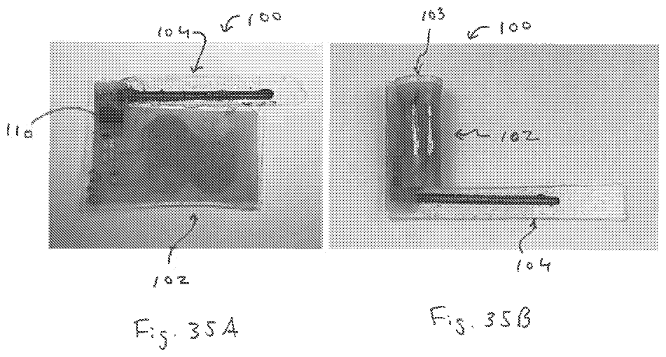

Both the circuitry, DHA, and FPS were coated with a thin, 0.5 mm layer of MED-4210 and allowed to cure at 60° C. As the MLX90129 RFIC was most raised from the device, the top layer silicone MED-4210 PDMS coating was not completely level, as shown in FIG. 34. After all MED-4210 was cured, the supportive plastic sheet was removed, and using a pair of scissors, a cut was made between the FPS and DHA allowing them to each roll independently, as shown in FIGS. 35A and 35B. Multiple devices were fabricated in this method, all with DHA diameter 8 mm spacing 15 mil. The DHA was then wrapped around a phantom blood flow system for experimental testing.

Experiments were conducted using a phantom blood flow system mimicking physiological blood flow in large caliber vessels, such as those used for hemodialysis vascular access. Blood-mimicking fluid with shear-thinning properties was used to reduce peak pulsatile pressure. A computer-controlled pulsatile pump system generated pulsatile flows, while pressure and flow sensors validated flow waveforms and systolic diastolic pressures.

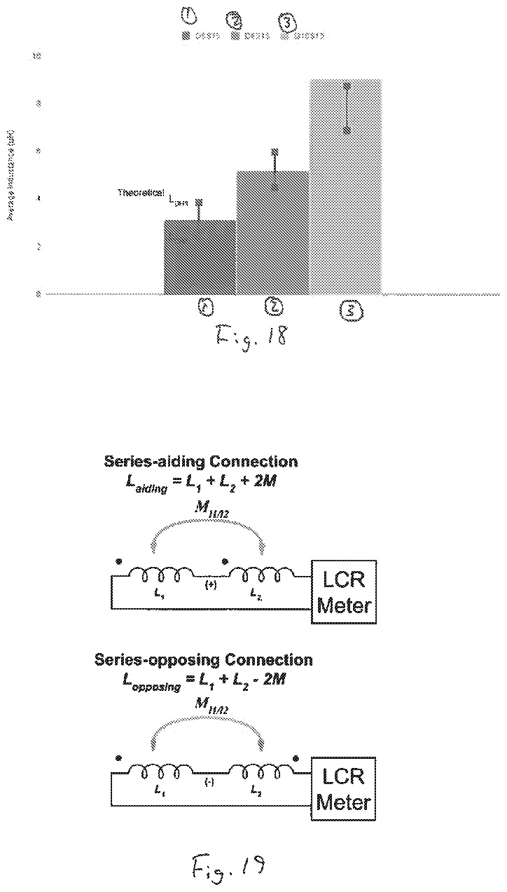

Blood vessel phantoms included a 6 mm diameter silicone tube (to simulate pulsation of a natural artery), and a 6 mm expanded poly-tetrafluoroethylene (ePTFE) graft designed for vascular access (GORE-TEX stretch graft). The FPS was wrapped around each phantom to detect blood flow; silicone phantoms were tested in air while ePTFE grafts were tested submerged in water (FIGS. 18C and 18D).

Vascular stenosis reduces the lumen diameter of blood vessels, reducing blood flow rate under constant systolic pressure conditions. To simulate this effect, both silicone and ePTFE vessel phantoms were sequentially narrowed using an adjustable vascular ligature clamp. Because the bench phantom was a single loop without collateral blood vessels, flow reductions also affected peak systolic pressure. To simulate physiology, therefore, the pump driving voltage was adjusted at each flow rate to maintain a constant systolic pressure of 120 mmHg.

In the first experiment, the FPS and DHA were wrapped around a silicone tube of 6 mm diameter mimicking an artery. Pulsations through the vessel stretched the FPS, and resistance changes were recorded by the RFID interface. Data were transferred from the DHA/FPS platform to a PC through the PN5180 RFID reader via a Teensy 4.1 development board and USB serial port.

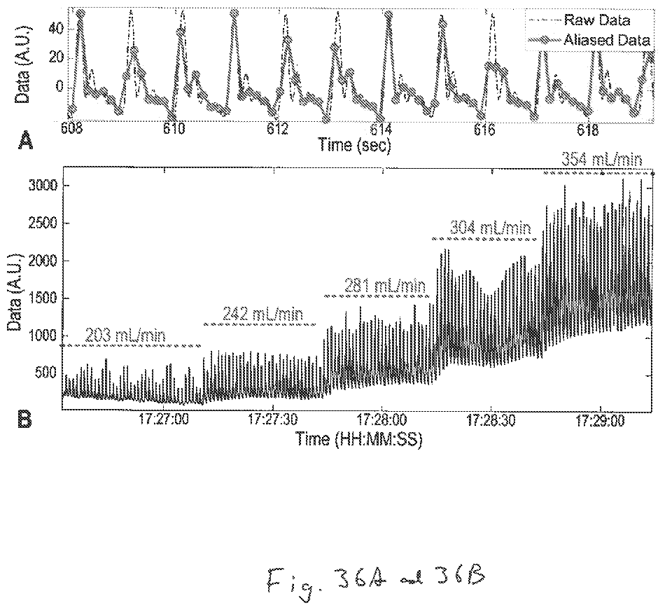

Due to the limited 12 Hz update rate of the PN5180 reader, hemodynamic signals from the FPS were under sampled. As a result, peak systolic pressures were not always captured. Experimentally, raw FPS signals from the artery phantom were recorded at 1,000 Hz, then decimated to the 12 Hz sample rate (FIG. 36A). Aliased data, as expected, showed inconsistent peak-to-peak amplitude. Average peak amplitude over 30-second periods, however, was relatively stable (as long as the pulsatile beats per minute and aliasing frequency of 12 Hz were not harmonically related). Therefore, data obtained by RFID readout were captured over windows at least 30 seconds long and analyzed by boxplots to compare median values while demonstrating the likely true peak-to-peak value without the aliasing artifact.

Raw recordings (FIG. 36B) from the sensor were processed in MATLAB R2021a to extract peak-to-peak amplitudes. Data were processed without pre-filtering using the findpeaks function to extract the amplitude of each peak. Peak amplitude distributions at each flow rate were extracted from 60-second recordings. Because the sensor signal was slightly undersampled (due to the limited 12 Hz update rate of the PN5180 reader) the true sensor signal peak in each pulsatile cycle was not always captured. Therefore, we analyzed sensor data by plotting the distribution of identified peaks, median values, and variance as boxplots.

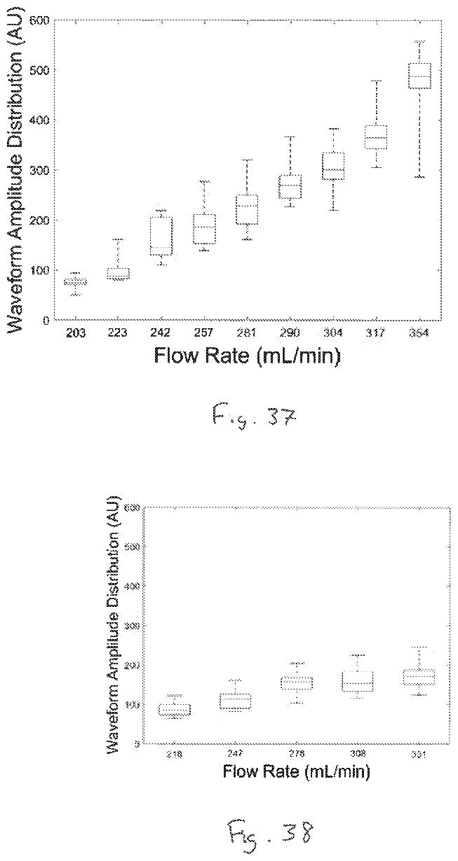

The distributions of extracted amplitudes for each flow rate showed a linear increase with flow rate (FIG. 37) over two trials. The monotonic response suggested the wireless FPS device could detect changes in vascular flow as low as 10 mL min.

The FPS was wrapped around a graft submerged in water in a tub to simulate being implanted inside the body. The DHA and RFID interface matching circuit was adjusted to account for DHA antenna detuning after submersion. The RFID reader was placed on the outside of the tub to simulate being on the skin surface. The diaphragm pump voltage was varied linearly to simulate increasing flow through the synthetic graft.

As before, sensor amplitudes increased monotonically with flow through the system (FIG. 38). Furthermore, because the ePTFE graft material had a lower elasticity than natural blood vessels (or silicone tubes) the overall sensor strain was lower when mounted on ePTFE. Despite the lower sensitivity, the system demonstrated detection of blood flow changes of about 30 mL/min using a flexible RIFD antenna and standard readout system.

This work demonstrated a flexible, implantable blood flow sensor for monitoring vessels or grafts. Research focused on the design of a cylindrical, hollow double-helix antenna (DHA) which can be flexed and curled to wrap around a blood vessel or graft during implantation. Additional research focused on implementing RFID protocol ISO15693 using a Melexis MLX90129 IC for wireless sensor readout using the DHA. A flexible pulsation sensor (FPS) was directly patterned onto the polyimide electronic interface to develop a fully functional prototype wireless sensor. Using a phantom blood flow system, further experiments showed that the sensor could detect fairly small changes in blood flow through a vessel or graft. Finally, while this prototype device was developed for vascular monitoring, the underlying technologies (DHA and FPS) may be adapted into many other flexible electronic sensor architectures.

Although the foregoing invention has been described in some detail by way of illustration and example for purposes of clarity of understanding, certain changes and modifications may be practiced within the scope of the appended claims.

What is claimed is:

1. An implantable blood flow sensor assembly for monitoring blood flow within a tubular structure within a body of a subject, comprising:
an antenna assembly;
a flexible sensor assembly having a cuff that is configured to positioned around a portion of the tubular structure within the body of the subject; and
an integrated circuit that is operably connected to the antenna assembly and the flexible sensor assembly.

2. The blood flow sensor assembly of claim 1, wherein the cuff of the flexible sensor is configured to be positioned around a portion of the vasculature or an implanted graft of the subject.

3. The blood flow sensor assembly of claim 2, the antenna assembly further comprising:
a flexible base layer;
a flexible top layer; and
a flexible printed circuit disposed between the base layer and the top layer,
wherein the flexible printed circuit includes a split-double helix antenna.

4. The blood flow sensor assembly of claim 3, wherein the split-double helix antenna is configured to be positioned around said portion of the tubular structure of the subject.

5. The blood flow sensor assembly of claim 4, wherein both the split-double helix antenna and the cuff of the flexible sensor assembly are configured to each form a cylinder about said portion of the tubular structure of the subject.

6. The blood flow sensor assembly of claim 5, wherein an inner diameter of the cylinder formed by both the split-double helix antenna and the cuff of the flexible sensor assembly is about 3.0 mm to about 10.0 mm in diameter.

7. The blood flow sensor assembly of claim 5, wherein the split-double helix antenna and the flexible sensor assembly are configured to be flexible independently of each other.

8. The blood flow sensor assembly of claim 5, wherein the flexible sensor assembly is configured to sense one of blood pressure or blood flow within the tubular structure.

9. The blood flow sensor assembly of claim 5, wherein the flexible sensor assembly comprises a flexible pulsation sensor that is configured to sense blood pressure of the subject.

10. The blood flow sensor assembly of claim 9, wherein the flexible pulsation sensor comprises a piezo resistive carbon black-polydimethylsiloxane nanocomposite.

11. The blood flow sensor assembly of claim 2, wherein the integrated circuit assembly includes a radio frequency integrated circuit.

12. A blood-flow sensor system for monitoring blood flow within a tubular structure within a body of a subject, comprising:
a transceiver assembly; and
a blood-flow sensor assembly comprising:
an antenna assembly; and
a flexible sensor assembly defining a cuff that is configured to be positioned around a portion of the tubular structure of the body,
wherein the blood flow sensor assembly is configured to be disposed entirely within the body of the subject, and the transceiver assembly is configured to communicate with the blood flow sensor assembly by way of a wireless communication.

13. The blood-flow sensor system of claim 12, the blood-flow sensor assembly further comprising an integrated circuit that is operably coupled to the antenna assembly and the flexible sensor assembly.

14. The blood-flow sensor system of claim 13, wherein the antenna assembly further comprises:
a flexible base layer;
a flexible top layer; and
a flexible printed circuit disposed between the base layer and the top layer,
wherein the flexible printed circuit includes a split-double helix antenna.

15. The blood flow sensor system of claim 14, wherein the split-double helix antenna is configured to be positioned around said portion of the tubular structure of the subject.

16. The blood flow sensor system of claim 15, wherein both the split-double helix antenna and the cuff of the flexible sensor assembly are configured to each form a cylinder about the portion of tubular structure of the subject.

17. The blood flow sensor system of claim 16, wherein the split-double helix antenna and the flexible sensor assembly are configured to be flexible independently of each other.

18. The blood flow sensor system of claim 16, wherein the flexible sensor assembly is configured to sense one of blood pressure or blood flow within the tubular structure.

19. The blood flow sensor system of claim 16, wherein the flexible sensor comprises a flexible pulsation sensor that is configured to sense blood pressure of the subject

20. The blood flow sensor of claim 8, wherein the flexible pulsation sensor comprises a piezo resistive carbon black-polydimethylsiloxane nanocomposite.

21. The blood flow sensor of claim 13, wherein the integrated circuit includes a radio frequency integrated circuit that is configured for wireless communication with the transceiver.
